(19) United States
(12) Patent Application Publication
(10) Pub. No.: US 2017/0150883 A1
(43) Pub. Date: Jun. 1, 2017

(54) LENS FOR INDICATING INTRAOCULAR PRESSURE OF EYE OF USER

(71) Applicant: HON HAI PRECISION INDUSTRY CO., LTD., New Taipei (TW)

(72) Inventor: Hsiu-Wen CHIEN, New Taipei (TW)

(21) Appl. No.: 15/066,407

(22) Filed: Mar. 10, 2016

(30) Foreign Application Priority Data
Nov. 26, 2015 (TW) 104139357

(51) Int. Cl. A61B 3/16; A61K 49/00; G02B 1/04

(52) U.S. Cl. CPC A61B 3/16; G02B 1/043; A61K 49/0063

(57) ABSTRACT

A lens wearable in an eye of a user can indicate intraocular pressure of the eye. The wearable lens includes a substrate containing anthocyanins. The substrate is formed by polymerization of a hydrophilic monomer and a hydrated polymer under the effects of a photoinitiator and a cross-linking agent. The anthocyanins in the substrate change color according to the pH of the eye, and the pH of the eye is determined according to the intraocular pressure of the eye.

### CROSS-REFERENCE TO RELATED APPLICATIONS

This application claims priority to Taiwanese Patent Application No. 104139357 filed on Nov. 26, 2015, the contents of which are incorporated by reference herein.

### FIELD

The subject matter herein generally relates to a lens for detecting intraocular pressure of an eye of a user of the lens.

### BACKGROUND

People affected by glaucoma often suffer from optic nerve damage and loss of vision. Signs of glaucoma generally include increased intraocular pressure (TOP) of the eye. Two of the most widely-used instruments for measuring IOP are the Goldmann tonometer and the Tono-Pen. The Tono-Pen is a microprocessor-controlled portable version of the Goldman tonometer. Both instruments may be invasive and uncomfortable for the user.

### DETAILED DESCRIPTION

Numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The description is not to be considered as limiting the scope of the embodiments described herein.

The term “comprising” means “including, but not necessarily limited to”; it specifically indicates open-ended inclusion or membership in a so-described combination, group, series and the like.

The present disclosure provides a lens wearable in an eye of a user. The lens can include a substrate. The substrate can contain anthocyanins. The lens can provide a way to detect intraocular pressure (TOP) of the eye of the user. In at least one embodiment, the lens can be a contact lens.

In at least one embodiment, the substrate can be made of at least one of hydrogel, silicone hydrogel, polysiloxane hydrogel, or phosphorus hydrogel. In at least one embodiment, the substrate is made of hydrogel. In at least one embodiment, the substrate can be formed by polymerization of a hydrophilic monomer and a hydrated polymer under the effects of a photoinitiator and a cross-linking agent. The hydrated polymer can serve as a backbone of the substrate. The hydrophilic monomer can increase a hydrophilic nature of the substrate and increase permeability of oxygen of the substrate. In at least one embodiment, the polymerization occurs under the condition of at least one of exposure to ultraviolet light and heating.

In at least one embodiment, the hydrophilic monomer is selected from at least one of hydroxyethyl methylacrylate, methyl methyl methacrylate, polydimethylsiloxane, 3-Methacryloxypropyletris(trimethyl siloxy)silane, N-vinylpyrrolidone, glycidyl methacrylate, N,N-dimethylacrylamide, or acrylic acid methyl ester.

In at least one embodiment, the hydrated polymer is selected from at least one of hydroxyethyl methylacrylate, methyl methacrylate, polydimethylsiloxane, or tri(hydroxymethyl)aminomethane.

In at least one embodiment, the cross-linking agent is selected from at least one of ethylene glycol dimethacrylate or trimethylolpropane trimethacrylate.

In at least one embodiment, the photoinitiator is selected from at least one of azodiisobutyronitrile or Irgacure-1173.

The anthocyanins are water-soluble vacuolar pigments that are capable of changing color in different pH environments. For example, when the pH is less than 7, the anthocyanins are red in color. When the pH is equal to 7, the anthocyanins are purple in color. When the pH is greater than 7, the anthocyanins are greenish-yellow in color. As the pH becomes more basic, the color density of the anthocyanins decreases.

In at least one embodiment, a mass percentage of the anthocyanins in the lens is between 0.0001% and 0.5%.

A normal range of TOP of the eye is between about 12 milligrams of mercury (mmHg) to about 22 mmHg, and a normal pH of the eye is about 7.4. Under normal circumstances, the TOP does not fall below 12 mmHg. The more the TOP exceeds 22 mmHg, the more the pH decreases. When the TOP is equal to about 70 mmHg, the pH of the eye is equal to about 7.03. The more the TOP exceeds 70 mmHg, the more the pH of the eye falls below 7.03.

In at least one embodiment, when liquid of the eye contacts the lens, the color of the lens changes according to the pH of the eye. Thus, the user can determine the TOP of the eye according to the color of the lens.

In another embodiment, the lens can serve as a pH detector in different applications besides detecting the TOP of the eye of a user. In another embodiment, the lens can serve as a colored contact lens for the user.

Anthocyanins are easily obtained from plant matter, so production of the lens is convenient and cheap.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size, and arrangement of the parts within the principles of the present disclosure, up to and including the full extent established by the broad general meaning of the terms used in the claims.

What is claimed is:

1. A lens configured to be worn in an eye of a user and configured to indicate intraocular pressure of the eye, the lens comprising a substrate containing anthocyanins therein.

2. The lens as in claim 1, wherein a mass percentage of the anthocyanins in the lens is between 0.0001% and 0.5%.

3. The lens as in claim 1, wherein the substrate is formed by polymerization of a hydrophilic monomer and a hydrated polymer under the effects of a photoinitiator and a cross-linking agent.

4. The lens as in claim 3, wherein the hydrophilic monomer is selected from at least one of hydroxyethyl methylacrylate, methyl methyl methacrylate, polydimethylsiloxane, 3-Methacryloxypropyletris(trimethylsiloxy)silane, N-vinylpyrrolidone, glycidyl methacrylate, N,N-dimethylacrylamide, or acrylic acid methyl ester.

5. The lens as in claim 3, wherein the hydrated polymer is selected from at least one of hydroxyethyl methylacrylate, methyl methacrylate, polydimethylsiloxane, or tri(hydroxymethyl)aminomethane.

6. The lens as in claim 3, wherein the cross-linking agent is selected from at least one of ethylene glycol dimethacrylate or trimethylolpropane trimethacrylate.

7. The lens as in claim 3, wherein the photoinitiator is selected from at least one of azodiisobutyronitrile or Irgacure-1173.

8. The lens as in claim 3, wherein the polymerization occurs under the condition of at least one of ultraviolet light and heating.

9. The lens as in claim 1, wherein the substrate is selected from at least one of hydrogel, silicone hydrogel, polysiloxane hydrogel, or phosphorus hydrogel.
